(19) United States
(12) Patent Application Publication
(10) Pub. No.: US 2003/0054413 A1
(43) Pub. Date: Mar. 20, 2003

(54) Bio-sensing platforms for detection and quantitation of biological molecules

(76) Inventors: Sriram Kumaraswamy, Santa Fe, NM (US); David Whitten, Santa Fe, NM (US); Duncan McBranch, Santa Fe, NM (US); Frauke Rininsland, Santa Fe, NM (US); Brent Arthur Burdick, Placitas, NM (US)

Correspondence Address: Supervisor, Patent Prosecution Services, PIPER RUDNICK LLP, 1200 Nineteenth Street, N.W., Washington, DC, 20036-2412, US

(21) Appl. No.: 10/226,300

(22) Filed: Aug. 23, 2002

Related U.S. Application Data
(60) Provisional application No. 60/314,094, filed on Aug. 23, 2001.

(51) Int. Cl. G01N 33/53; C08B 37/00

(52) U.S. Cl. 435/7.5; 536/18.7; 525/256

(57) ABSTRACT

A bioconjugate comprising a fluorescer (P) linked to a quencher (Q) by a tether (T) is provided. The tether (T) includes a segment that can recognize and interact with a target biomolecule. In the absence of a specific interaction of the bioconjugate with an enzyme or other target biomolecule recognizing the bioconjugate, the fluorescence of the polymer (P) is attenuated or modified by the relatively close proximity thereto of the quencher (Q). As a consequence of the association of the bioconjugate with the target biomolecule, a reaction can occur which results in a cleavage of the bioconjugate tether and a release of the fluorescent polymer and/or quencher. This sequence of events can be followed by an enhancement or amplification of the polymer fluorescence.

This application claims priority from U.S. Provisional Application No. 60/314,094, filed Aug. 23, 2001, which is herein incorporated by reference in its entirety.

### BACKGROUND OF THE INVENTION

1. Field of the Invention

The present invention relates generally to molecular sensors for detecting molecular interactions. In particular, the present invention relates to bioconjugates comprising a quencher (Q), a fluorescer (P) and a tether (T) linking the fluorescent polymer with the quencher (i.e., QTP) wherein the fluorescer (P) comprises a plurality of associated fluorescent species (e.g., a fluorescent polymer, oligomer or “virtual polymer”) and the tether comprises a segment capable of recognizing and interacting with a target biomolecule. The bioconjugate can be used for detection and quantitation of biological molecules such as nucleic acids and enzymes.

2. Discussion of the Background

The enzyme linked immunosorbant assay (i.e., ELISA) is the most widely used and accepted technique for identifying the presence and biological activity of a wide range of proteins, antibodies, cells, viruses, etc. An ELISA is a multi-step “sandwich assay” in which the analyte biomolecule is first bound to an antibody attached to a surface. A second antibody then binds to the biomolecule. In some cases, the second antibody is attached to a catalytic enzyme which subsequently “develops” an amplifying reaction. In other cases, this second antibody is biotinylated to bind a third protein (e.g., avidin or streptavidin). This protein is attached either to an enzyme, which creates a chemical cascade for an amplified calorimetric change, or to a fluorophore for fluorescent tagging.

Despite its wide use, there are many disadvantages to ELISA. For example, because the multi-step procedure requires both precise control over reagents and development time, it is time-consuming and prone to “false positives”. Further, careful washing is required to remove nonspecific adsorbed reagents.

Fluorescence resonance energy transfer (i.e., FRET) techniques have been applied to both polymerase chain reaction-based (PCT) gene sequencing and immunoassays. FRET uses homogeneous binding of an analyte biomolecule to activate the fluorescence of a dye that is quenched in the off-state. In a typical example of FRET technology, a fluorescent dye is linked to an antibody (F-Ab), and this diad is bound to an antigen linked to a quencher (Ag-Q). The bound complex (F-Ab:Ag-Q) is quenched (i.e., non-fluorescent) by energy transfer. In the presence of identical analyte antigens which are untethered to Q (Ag), the Ag-Q diads are displaced quantitatively as determined by the equilibrium binding probability determined by the relative concentrations, [Ag-Q]/[Ag]. This limits the FRET technique to a quantitative assay where the antigen is already well-characterized, and the chemistry to link the antigen to Q must be worked out for each new case.

Other FRET substrates and assays are disclosed in U.S. Pat. No. 6,291,201 as well as the following articles: Anne et al., “High Throughput Fluorogenic Assay for Determination of Botulinum Type B Neurotoxin Protease Activity”, Analytical Biochemistry, 291, 253-261 (2001); Cummings et al., A Peptide Based Fluorescence Resonance Energy Transfer Assay for Bacillus Anthracis Lethal Factor Protease”, Proc. Natl. Acad. Scie. 99, 6603-6606 (2002); and Mock et al., “Progress in Rapid Screening of Bacillus Anthracis Lethal Activity Factor”, Proc. Natl. Acad. Sci. 99, 6527-6529 (2002).

Other assays employing intramolecularly quenched fluorescent substrates are disclosed in the following articles: Zhong et al., Development of an Internally Quenched Fluorescent Substrate for Escherichia Coli Leader Peptidase”, Analytical Biochemistry 255, 66-73 (1998); Rosse et al., Rapid Identification of Substrates for Novel Proteases Using a Combinatorial Peptide Library, J. Comb. Chem. 2000, 2, 461-466; and Thompson et al., “A BODIPY Fluorescent Microplate Assay for Measuring Activity of Calpains and Other Proteases”, Analytical Biochemistry, 279, 170-178 (2000). Assays have also been developed wherein changes in fluorescent polarization have been measured and used to quantify the amount of an analyte. See, for example, Levine et al., “Measurement of Specific Protease Activity Utilizing Fluorescence Polarization”, Analytical Biochemistry 247, 83-88 (1997). See also Schade et al., BODIPY-α-Casein, a pH-Independent Protein Substrate for Protease Assays Using Fluorescence Polarization”, Analytical Biochemistry 243, 1-7 (1996).

There still exists a need, however, to rapidly and accurately detect and quantify biologically relevant molecules with high sensitivity.

### BRIEF DESCRIPTION OF THE DRAWINGS

The present invention may be better understood with reference to the accompanying drawings in which:

FIG. 1A shows a QTP bioconjugate for nucleic acid detection according to the invention wherein the quencher and fluorescer are each located on segments within the tether;

FIG. 1B shows a QTP bioconjugate for nucleic acid detection according to the invention wherein the quencher and fluorescer are located at opposite ends of the tether;

FIG. 1C illustrates an assay according to the invention wherein a QTP bioconjugate as shown in FIG. 1A is used to detect the presence and/or amount of a nucleic acid target in a sample;

FIG. 2A shows a QTP bioconjugate for enzyme detection according to the invention wherein the quencher and oligomer are each located on segments within a peptide or carbohydrate tether;

FIG. 2B shows a QTP bioconjugate for enzyme detection according to the invention wherein the quencher and oligomer are located at opposite ends of the peptide or carbohydrate tether;

FIG. 2C illustrates an assay for enzyme detection according to the invention wherein a QTP bioconjugate as shown in FIG. 2A is used to detect the presence and/or amount of a cleavage enzyme in a sample;

FIG. 2D illustrates an assay for enzyme detection according to the invention wherein a QTP bioconjugate as shown in FIG. 2A is used to detect the presence and/or amount of a transferase enzyme in a sample;

FIG. 3 is an illustration of an assay according to the invention wherein the fluorescer comprises a plurality of fluorescent species associated with a microsphere and wherein the quencher comprises a plurality of quencher moieties conjugated to the surface of the microsphere through a tether segment capable of recognizing and interacting with a target biomolecule;

FIG. 4 shows the structure of a biotinylated anionic conjugated polymer (i.e., PPE-B) which can be bound to the surface of a solid support via biotin/streptavidin associations and used as a fluorescer according to the invention;

FIG. 5 shows the structure of a QTB bioconjugate according to the invention comprising a quencher and a biotin molecule conjugated to a tether which can be used in an assay for enterokinase;

FIG. 6A illustrates an assay according to the invention wherein a sample containing an enzyme is incubated with a QTB bioconjugate prior to contacting the incubated mixture with a fluorescer;

FIG. 6B illustrates a control for the assay of FIG. 6A wherein no QTB reactant is added to the sample containing the enzyme prior to contacting the sample with the fluorescer;

FIG. 6C illustrates a control for the assay of FIG. 6A wherein no enzyme is added to the sample prior to contacting the sample with the fluorescer;

FIG. 7A shows an avidin core binding cassette which can be used to synthesize a QTP bioconjugate according to the invention; and

FIG. 7B illustrates the synthesis of a QTP bioconjugate using the avidin core binding cassette of FIG. 7A and the use of this bioconjugate in an assay.

### SUMMARY OF THE INVENTION

According to a first aspect of the invention, a bioconjugate is provided. The bioconjugate includes: a tether comprising a segment capable of recognizing and interacting with a target biomolecule; a fluorescer comprising a plurality of associated fluorescent species, the fluorescer conjugated to a first location on the tether; and a quencher for the fluorescer conjugated to a second location on the tether; wherein the segment capable of recognizing and interacting with the target biomolecule is located between the first and second locations on the tether. The segment capable of recognizing and interacting with the target biomolecule can comprise a peptide or nucleic acid sequence. The fluorescer can be a polymer or oligomer comprising a plurality of fluorescent repeating units. Alternatively, the fluorescer can be a solid support associated with a plurality of fluorescent species.

According to a second aspect of the invention, a method of assaying for the presence and/or amount of a target analyte in a sample is provided. The method comprises: incubating the sample with a bioconjugate as set forth above; and measuring the fluorescence of the incubated sample; wherein the measured fluorescence of the incubated sample is an indication of the presence and/or the amount of the target analyte in the sample.

According to a third aspect of the invention, a method of assaying for the presence or amount of a target analyte in a sample is provided. The method comprises: incubating the sample with a bioconjugate comprising a quencher and a reactive group conjugated to a tether at first and second locations respectively, wherein the tether comprises a segment between the first and second locations capable of recognizing and interacting with the target analyte; adding a fluorescer to the incubated sample to form a sample mixture, the fluorescer comprising a solid support associated with a plurality of fluorescent species, wherein the solid support comprises surface functional groups reactive with the reactive group on the bioconjugate such that the bioconjugate can be attached to the solid support, the attachment of the bioconjugate to the solid support quenching the fluorescence of the fluorescer; allowing the reactive group on the bioconjugate to react with the surface functional groups on the solid support; and subsequently measuring the fluorescence of the sample mixture; wherein the amount of fluorescence of the sample mixture indicates the presence and/or amount of the target analyte in the sample.

According to a fourth aspect of the invention, an assay for an intracellular target analyte is provided. The assay comprises: transfecting a cell with a bioconjugate as set forth above wherein the tether comprises a segment capable of recognizing and interacting with an intracellular target biomolecule; and measuring the fluorescence of the cell. The measured fluorescence of the cell indicates the presence and/or amount of the target biomolecule in the cell.

According to a fifth aspect of the invention, a biotinylated fluorescer comprising a plurality of associated fluorescent species is provided. The biotinylated fluorescer can be a biotinylated fluorescent polymer or oligomer such as a biotinylated poly(phenylene ethynylene) polymer. The biotinylated fluorescer can also be a biotinylated solid support having a plurality of fluorescent species associated therewith, wherein the solid support comprises biotin groups available for reaction.

According to a sixth aspect of the invention, a quenching reagent is provided. The quenching reagent comprises: a tether comprising a segment capable of recognizing and interacting with a target biomolecule; a quencher conjugated to a first location on the tether, the quencher capable of quenching the fluorescence of a fluorescer comprising a plurality of associated fluorescent species; and a biotin molecule conjugated to a second location on the quencher. According to this aspect of the invention, the segment capable of recognizing and interacting with the target biomolecule is located between the first and second locations on the tether. The segment capable of recognizing and interacting with a target biomolecule can comprise a peptide sequence such as (Asp)₄Lys. The quencher can be QSY-7.

According to a sixth aspect of the invention, a bioconjugate is provided. The bioconjugate comprises: a tether comprising a segment capable of recognizing and interacting with a target biomolecule; a first avidin molecule conjugated to a first location on the tether; and a second avidin molecule conjugated to a second location on the tether. According to this aspect of the invention, the segment capable of recognizing and interacting with the target biomolecule is located between the first and second locations on the tether. The bioconjugate can further comprise: a fluorescer comprising a plurality of associated fluorescent species conjugated to the first avidin molecule; and a quencher capable of quenching the fluorescence of the fluorescer conjugated to the second avidin molecule. The fluorescer can be a solid support (e.g., a microsphere or bead) having a plurality of fluorescent species associated therewith.

### DESCRIPTION OF THE PREFERRED EMBODIMENTS

Bioconjugates comprising a ligand (L) for a target biological molecule tethered (T) to a quencher (Q) that associates with and quenches a fluorescent polymer (P) are disclosed in U.S. patent application Ser. No. 09/850,074, herein incorporated by reference in its entirety. These bioconjugates (designated “QTL bioconjugates”) take advantage of super-quenching of fluorescent polyelectrolytes by, for example, electron transfer or energy transfer quenching. A fluorescent polymer (P) can form an association complex with a QTL bioconjugate, usually one with a charge opposite that of the fluorescent polymer. The QTL bioconjugate includes a quencher (Q) linked through a covalent tether to a ligand (L) that is specific for a particular biomolecule. The association of the ligand of the QTL bioconjugate with the biomolecule either separates the QTL bioconjugate from the fluorescent polymer, or modifies its quenching in a readily detectable way, thus allowing sensing of the biomolecule by a change in fluorescence. In this manner, the biomolecule can be detected at very low concentrations.

The present invention is directed to a bioconjugate comprising a reactive tether (i.e., a tether comprising a segment capable of recognizing and interacting with a target biomolecule) linking a fluorescer (P) with a quencher (Q) for the fluorescer. This bioconjugate is hereinafter referred to as a “QTP bioconjugate”. The QTP bioconjugate is therefore a reactive molecular sensor that includes a quencher (Q) linked via a reactive tether (T) to a fluorescer (P) (e.g., a fluorescent polymer or oligomer). In the absence of a specific association of the tether of the QTP bioconjugate with an enzyme or other target biomolecule, the fluorescence of P is attenuated or modified by the relatively close proximity of Q.

According to the invention, the reactive tether (T) can recognize and associate with a target biomolecule. As a consequence of the association of the QTP molecule with the target biomolecule, a reaction can occur which results in a cleavage of the QTP bioconjugate and the release of free P and/or Q. The target biomolecule can itself be an enzyme which cleaves the tether. Alternatively, the target biomolecule can be a biomolecule (e.g., a nucleic acid) which, when hybridized to the tether, allows the tether to be cleaved by an enzyme. This sequence of events can result in an enhancement of fluorescence. In certain embodiments, the reaction may be catalytic. Thus, amplification of the fluorescence response may occur.

Cleavable QTP (Quencher-Tether-Polymer) Substrates for Biodetection and Bioassay.

The bioconjugate according to the invention combines a fluorescer (P) whose emission is subject to quenching (e.g., super-quenching) by a quencher component (Q) that extinguish the fluorescence of P (e.g., by energy transfer or electron transfer quenching). Although the fluorescer is referred to herein as a polymer (P), it should be understood that other fluorescers can also be used including, but not limited to, oligomers, polymer segments or virtual polymers. Moreover, the fluorescer according to the invention comprises a plurality of associated fluorescent species (i.e., a plurality of fluorescent species associated with one another). The fluorescent species of the fluorescer can be associated with one another in the form of a polymer or oligomer comprising a plurality of fluorescent repeating units.

Alternatively, the fluorescent species can be associated with one another through attachment to a solid support. Exemplary solid supports suitable for use in the present invention include: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes. The fluorescent polymer or oligomer can be associated with the solid support by any suitable means including, but not limited to: covalent attachment to the solid support; adsorption onto the surface of the solid support; or via interactions between a biotin moiety (e.g., on a fluorescent polymer or oligomer) and an avidin, neutravidin or streptavidin moiety on the solid support surface.

Exemplary fluorescers according to the invention include, but are not limited to: conjugated polyelectrolytes; biotinylated conjugated polyelectrolytes; functionalized conjugated oligomers; charged conjugated polymers; uncharged conjugated polymers; conjugated polymer blends; and J-aggregated polymer assembly comprising assembled monomers or oligomers. According to a preferred embodiment of the invention, the fluorescer can be constructed from an oligosaccharide.

According to a preferred embodiment of the invention, the fluorescer can be a “virtual polymer” obtained by assembling monomeric or oligomeric components on the surface of a particle or other support such that excitonic interactions between the assembled components facilitates amplified super-quenching compared to the individual components. According to a preferred embodiment of the invention, a QTP bioconjugate substrate comprising a virtual polymer may be constructed from a mixture of a quencher-tether-biotin conjugate (i.e., a “QTB” molecule) and a fluorescer (e.g., a biotinylated fluorescent polymer). The fluorescer and the “QTB” molecule can be attached to the surface of a solid support (e.g., a bead or microsphere) to form a QTP bioconjugate according to the invention.

Fluorescers (e.g., fluorescent oligomers, polymers and “virtual polymers”) suitable for use in the present invention are disclosed in copending U.S. patent application Ser. No. 10/098,387, which application is incorporated herein by reference in its entirety.

If the fluorescer component of a QTP bioconjugate is anchored on a support containing an array of un-functionalized fluorescent polymers (or oligomers), the unreacted QTP molecule may exhibit surface activated super-quenching in the ensemble. Cleavage of the QTP molecule with the concurrent expulsion of the quencher may afford additional amplification by “turning on” the fluorescence of the entire polymer ensemble.

According to one embodiment of the present invention, the fluorescer and quencher components of the QTP bioconjugate are linked together by a tether, T, which contains a segment which is selectively cleavable by an enzyme-catalyzed reaction. A segment of the tether (T) can include, for example, both a recognition element that associates with a target biomolecule and a cleavage site that reacts when the QTP is associated with the target biomolecule. The target biomolecule may therefore either promote or catalyze the cleavage of QTP. Alternatively, the cleavage may be initiated by an additional enzyme (or through single or multiple enzyme cofactors).

Ultrasensitive Nucleic Acid Detection Using QTP (Quencher-Tether-Polymer) Based Superquenching.

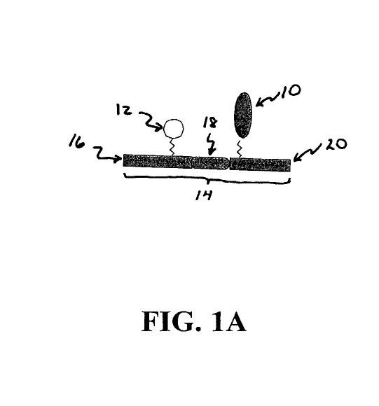

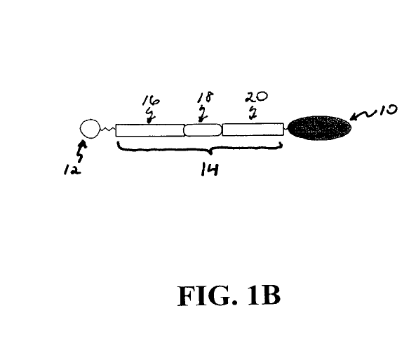

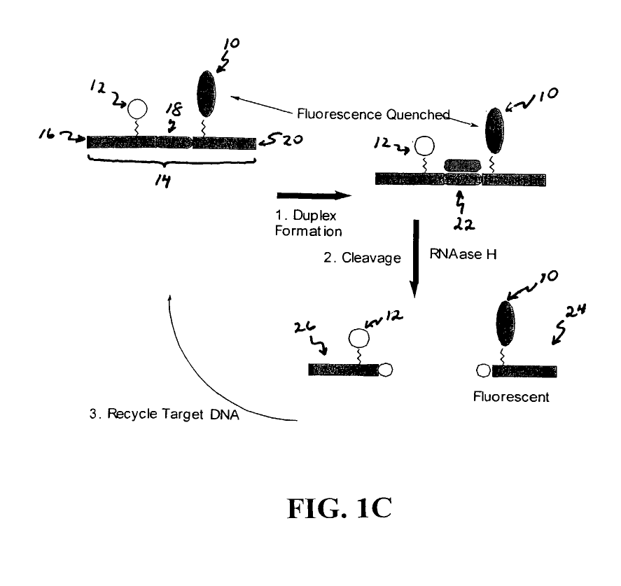

The following example combines the use of fluorescent polymer super-quenching with cycling probe technology (CPT). Cycling probe technology is disclosed, for example, by Duck et al., Biotechniques, 9, 142-149 (1990). As shown in FIGS. 1A-1C, the bioconjugate used in this Example is a synthetic bioconjugate that includes a fluorescer 10 (i.e., an oligomer, polymer or virtual polymer) linked covalently to a quencher 12 (Q) through a recognition strand 14 (T) that includes the following components: a sequence of DNA or Peptide Nucleic Acid (PNA) 16; a segment of RNA 18; and a second DNA or PNA segment 20. According to this embodiment of the invention, the segment of RNA 18 comprises a sequence complementary to a target DNA. In the absence of hybridization of the segment of RNA 18 with the target DNA, the fluorescence of the polymer is quenched.

As shown schematically in FIG. 1A, polymer component 10 and quencher 12 may be located on segments within the strand 14. Alternatively, as shown in FIG. 1B, the polymer component 10 and quencher 12 may be located at either end of strand 14. The quencher can be an energy transfer or electron transfer quencher. However, given the relatively large separation between the quencher and the polymer, an energy transfer quencher is generally preferable.

The QTP molecule, although it contains an RNA segment, is not cleaved in the presence of Ribonuclease H (i.e., RNaseH), an enzyme that specifically digests RNA only when hybridized to a nucleic acid (e.g., in an RNA:DNA duplex). In the presence of target DNA, however, hybridization of the nucleic acid segment of the QTP bioconjugate with the target leads to duplex formation 22 as shown in FIG. 1C. Therefore, the RNA segment of the bioconjugate can be digested by RNaseH only when hybridized to a target nucleic acid. The resulting cleavage of the tether of the QTP bioconjugate results in a separation of the quencher (Q) from the polymer (P) producing a quencher containing fragment 26 and a fluorescer containing fragment 24. This separation can result in a change in fluorescence.

Since only the RNA of the QTP molecule is cleaved, the target nucleic acid may be recycled to hybridize with additional QTP molecules, thus affording an amplification in which one molecule of target DNA can be used to effect the release of multiple polymer fluorophores and/or quenchers. Although the cycling probe amplification as shown in FIG. 1C results in an accumulation of both quencher-DNA (or PNA) and polymer-DNA (or PNA) fragments 24, 26, significant intermolecular quenching of the fluorescence can be averted by “tuning” the charge on these fragments so that there is net overall repulsion between the fragments generated. Thus, the QTP bioconjugate can be tailored such that the Q and P containing fragments 24, 26 are oppositely charged after cleavage of the tether.

The example presented above can also be used with RNA targets through a prior reverse transcription step. This embodiment of the invention is particularly attractive for diagnostic applications in that it offers sensitivity equivalent to polymerase chain reaction (PCR) in a format that is much simpler to use and to automate (e.g., isothermal, homogeneous operation). As an additional advantage, since the process outlined in FIG. 1C involves a linear amplification scheme, the process inherently lends itself to internal calibration and quantification of target DNA (or RNA), which is extremely useful in areas where quantitative outputs are desired such as with viral load testing.

QTP Assays for Protease, Glycosidase and Transferase Enzymes

Synthetic QTP bioconjugates wherein the “tether” component is constructed from a polypeptide can be used to sense enzymes that can cleave peptide or other scissile linkages in the tether. The sensing may be tailored by synthesis to be specific, for example, to a single protease, or to be general for a broader family of enzymes.

As discussed below, the use of specific fluorescers can also provide a means of delivery of a QTP molecule into a cell or, alternatively, a means for anchoring the QTP molecule on a support or membrane. In the case of simple protease enzymes, the use of a QTP bioconjugate can provide sensing with the same type of amplification outlined above.

While the examples presented below involve a specific protease (i.e., enterokinase), it should be understood that assays according to the invention can be extended to any target of general or specific interest including, but not limited to, anthrax lethal factor, botulinum type B neurotoxin, caspase enzymes or retroviral proteases.

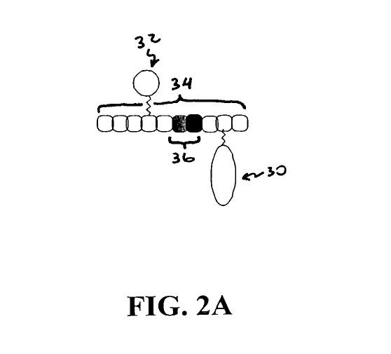

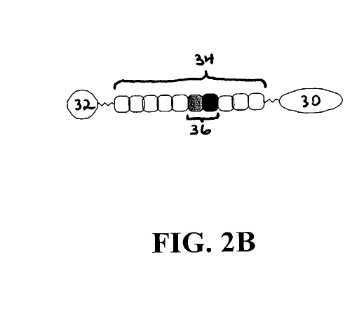

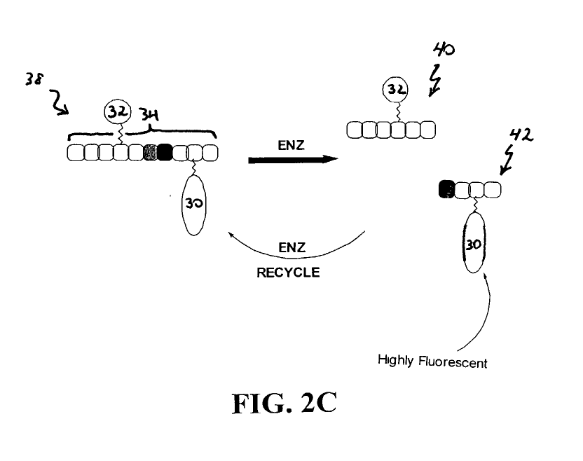

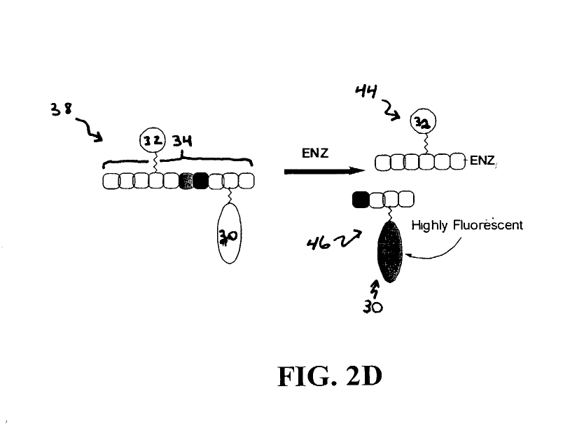

QTP bioconjugates having a tether comprising a peptide or carbohydrate segment are illustrated in FIGS. 2A and 2B. As shown in FIG. 2A, the quencher 30 and fluorescer 32 can be located on units within the tether 34. Alternatively, as shown in FIG. 2B, quencher 30 and fluorescer 32 may be located at either the end of the tether 34. In either case, a cleavage site 36 on the tether is located between the points of attachment of the fluorescer and quencher.

Assays employing a bioconjugate as shown in FIG. 2A are illustrated in FIGS. 2C and 2D for cleavage and transferase enzymes, respectively. As shown in FIG. 2C, an enzyme, designated ENZ (e.g., a protease) can be used to cleave the tether of the QTP bioconjugate 38. Since the enzyme can function catalytically to cleave multiple QTP bioconjugates 38, amplification of the already highly sensitive fluorescence detection afforded by the “release” of the super-quenching can be amplified. As shown in FIG. 2A, cleavage of the tether results in the production of quencher containing and fluorescer containing fragments 40, 42. As with the assay illustrated in FIG. 1C, significant intermolecular quenching of the fluorescence can be avoided by “tuning” the charge on these fragments so that there is net overall repulsion between the fragments generated.

FIG. 2D illustrates cleavage of the tether with a transferase enzyme (designated ENZ). As shown in FIG. 2D, the transferase enzyme cleaves the tether and receives the quencher fragment 44 of the QTP bioconjugate 38. A fluorescer fragment 46 is also shown. However, the QTP bioconjugate can be engineered synthetically so that, upon reaction of the QTP bioconjugate with the transferase enzyme, the transferase enzyme will receive either the quencher fragment or the fluorescer fragment of the cleaved QTP bioconjugate. In either case, the transferase may be inactivated by the process, and sensed simultaneously. This provides an example of a “killer-sensor”, a sensor that neutralizes a reactive molecule, activates a secondary chemical process, or initiates a catalytic chemical cascade.

According to a further embodiment of the invention, QTP bioconjugates can be synthesized having a tether constructed from an oligosaccharide or glycoconjugate containing an appropriate substrate binding and cleavage site thus making it possible to sense enzymes, such as glycosidases or transferases, that can cleave a specific glycoside linkage yielding either hydrolysis or transfer products. These QTP bioconjugates can therefore be used in processes similar to those described above for peptide-based QTP bioconjugates and illustrated in FIGS. 2C and 2D. One particularly useful application of monitoring transferase enzyme activity by this scheme would be assays for the family of tyrosine kinases, either in an extracellular or intracellular mode.

Tailoring of the Fluorescent Polymer to Permit Intracellular Assay.

The polymer (or oligomer) component of a QTP molecule may be constructed from either a conjugated polymer segment or a segment in which individual chromophores are not directly conjugated but interact via specific aggregation effects. As has been shown for cyanine-derivatized poly-L-lysine, the conformations of fluorescent polymers or oligomers may be controlled by the building block onto which the chromophores are attached. Cyanine-derivatized poly-L-lysine has been found to adopt a predominantly beta sheet structure. Due to the conformational similarity between the synthetic dye-derivatized polypeptides and natural peptides, fluorescent oligomers or polymers can be used as a delivery vehicle to bring QTP bioconjugates according to the invention into a cell for tailored intracellular assays. In a similar manner, QTP bioconjugates comprising fluorescent polymers or oligomers constructed from functionalized oligosaccharides can also serve as delivery vehicles to bring the molecular sensor to a membrane or cell surface and subsequently into a cell.

An example of the utility of this approach is a QTP bioconjugate constructed to serve as a substrate for a series of caspase (proteolytic) enzymes, the presence of which intracellularly indicates the initiation of apoptosis. The ability to monitor the initiation or supression of apoptosis may provide a diagnostic tool to assess cell death (as in AIDS) or the proliferation of cells, including, but not limited to, examples such as Human Pappilloma Virus or B cell lymphoma.

Assay for Endoproteases

All of the following examples utilize the enzyme enterokinase, which is a prototype protease. In the examples, the enterokinase cleaved polypeptide sequence of (Asp)₄Lys was incorporated into the tether component of a molecule comprising biotin tethered to a quencher (hereinafter referred to as a “QTB reagent”). This reagent can be readily linked, for example, to a solid support bearing free streptavidin sites via biotin-streptavidin association. According to a preferred embodiment of the invention, the reagent can be linked to a bead-supported poly(phenylene ethynylene) (e.g, PPE-B) fluorescent polymer. When linked to the bead, fluorescence from the poly(phenylene ethynylene) on the solid support (e.g., the bead) can be quenched (i.e., super-quenched) due to the close proximity of the tethered quencher (Q). When the tether is cleaved, however, the quencher is freed and, consequently, quenching can be reduced.

### EXAMPLE 1

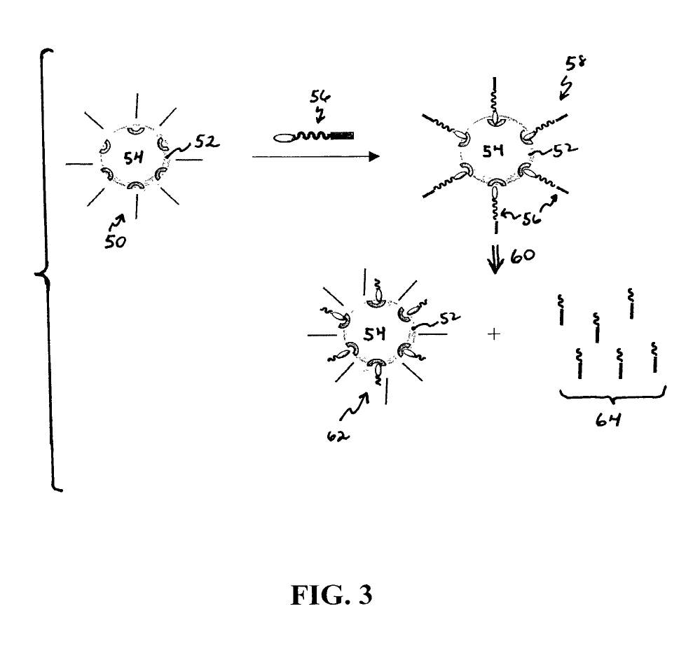

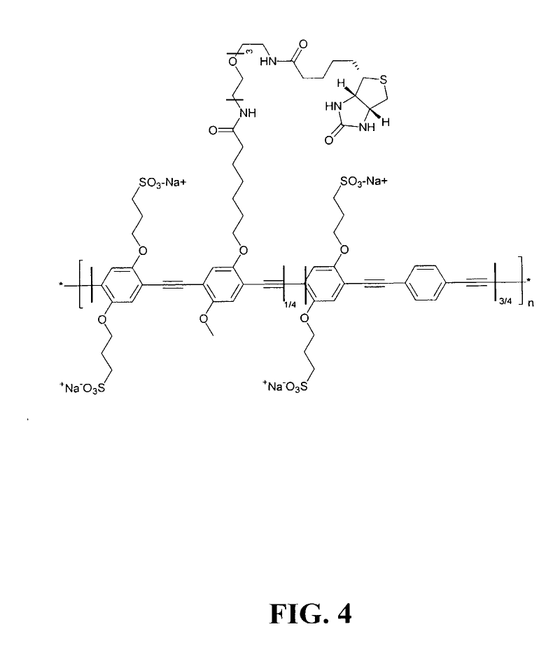

This assay is illustrated in FIG. 3. Commercial polystyrene beads 54 containing streptavidin covalently tethered to the surface (0.46 micron microspheres purchased from Bangs Laboratories, Inc., Fishers, Id.) were coated with a fluorescent species 52 (e.g., biotinylated anionic conjugated polymer PPE-B as shown in FIG. 4) to form fluorescer coated beads 50. The PPE-B polymer shown in FIG. 4 is a biotinylated derivative of poly(phenylene ethynylene) (PPE).

According to the invention, the level of loading of fluorescent species 52 on the surface of beads 54 can be controlled. The number of available biotin binding sites (maximum biotin—FITC binding capacity=1.35 mg/mg of microspheres) is also variable and can be controlled according to the invention.

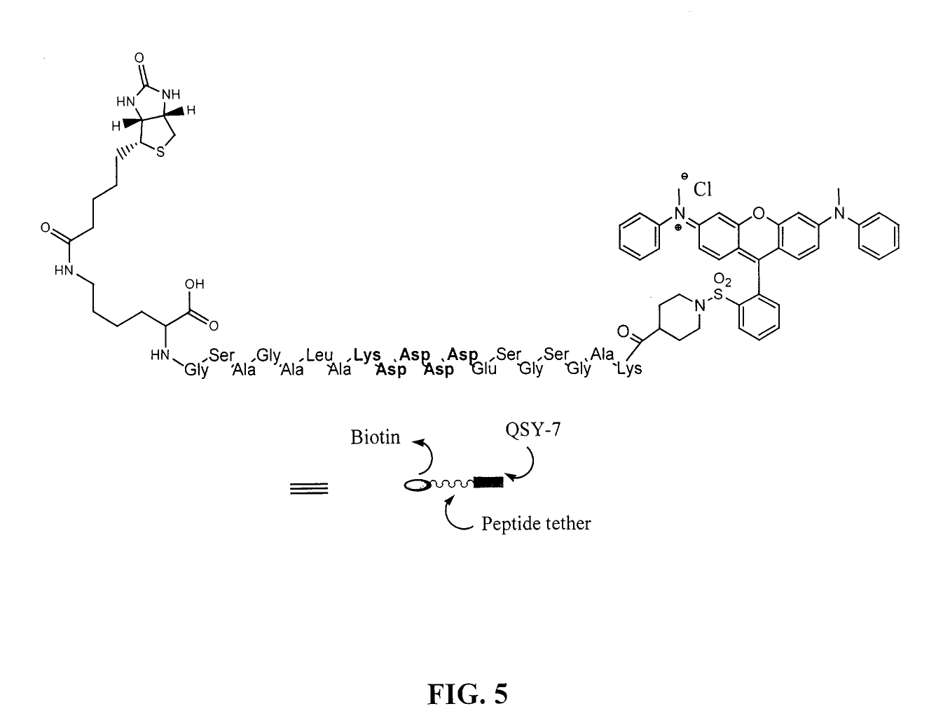

In the Example, 2 mL of a 4 mM solution of a QTB reagent 56 having the structure shown in FIG. 5 were added to a 0.9 mL suspension of PPE-B coated streptavidin microspheres (concentration=1.63e10 microspheres/mL) in water. The molecule shown in FIG. 5 comprises a quencher (i.e., QSY-7) linked via a peptide tether to a biotin moiety. This amount of QTB reagent was approximately a 10 fold excess based on the number of free biotin binding sites available on the microspheres. The suspension was shaken gently on a vortexer for 2 hours at room temperature. The mixture was then centrifuged at 13,000 rpm at 20° C. for 60 minutes to pellet the microspheres. The supernatant liquid was carefully removed and replaced with fresh water. The residue was then re-suspended by gentle vortexing. The wash procedure involving alternate centrifugation/resuspension steps which were repeated four times to ensure complete removal of all loosely bound and unbound QTB. The cleaned microspheres 58 were finally resuspended in 1 mL of water to afford the stock QTP suspension.

The effect of binding QTB to PPE-bound streptavidin microspheres on the emission intensity of the PPE-B chromophores was then probed in a fluorimeter. It was found that the QTB microspheres had lost about 80% of their original fluorescence due to energy transfer quenching by the bound QSY-7 chromophores.

As shown in FIG. 3, treatment of the QTP suspension with enterokinase results in the cleavage 60 of the tethers and the production of quencher fragments 64 and a fluorescer fragment 62. As a result of separation of the fragments 62 and 64, a change in the fluorescence of the fluorescer fragment 62 can occur.

### EXAMPLE 2

A more rapid assay was achieved by treating an aqueous solution of the QTB with enterokinase in a homogeneous reaction. Hydrolysis of the QTB was complete in thirty minutes when a 0.53 mM solution was reacted with 0.1 U of EKMax (EKMax is a clone of the catalytic subunit of enterokinase enzyme and is supplied by Invitogen Inc., Carlsbad, Calif.). One (1) Unit is defined as the amount of EKMax that will digest 20 mg of thioredoxin-chloramphenicol acetyl transferase fusion protein to 90% completion in 16 hours at 37° C. in 50 mM Tris-HCl, pH 8.0, mM CaCl₂, and 0.1% Tween-20 (IX EKMax Buffer). Adding a constant amount of a PPE-B bound streptavidin microsphere suspension to aliquots of the reaction solution at various time intervals monitored the progress of the reaction. The quench of polymer fluorescence was strong initially and reduced in degree with increasing amounts of peptide hydrolysis resulting finally in complete fluorescence recovery when hydrolysis was complete.

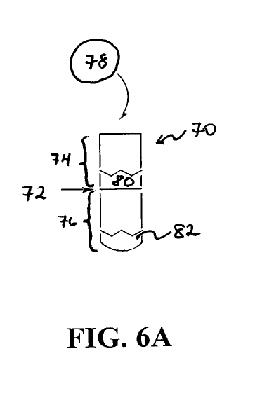

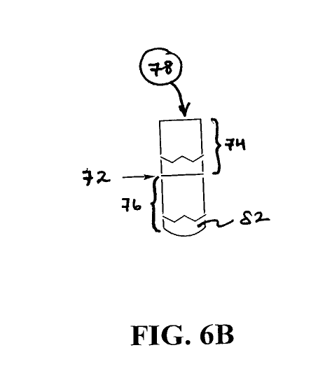

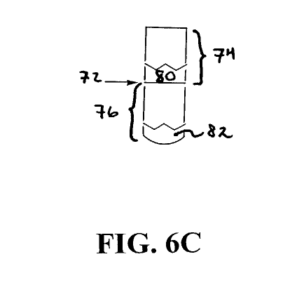

A schematic of the use of the above procedure in a practical assay is shown in FIGS. 6A-6C. A two-compartment container 70 (e.g., a cuvet) with a breakseal or removable divider 72 between the two compartments 74 and 76 is shown in FIG. 6A. In an assay according to the invention, an enzyme solution 78 (i.e., the analyte) is added to the top compartment and allowed to react and incubate with a QTB reagent 80. As shown in FIG. 6A, the bottom compartment 76 contains a solution comprising PPE-B coated beads 82. Removal of the divider 72 (e.g., breakseal) allows mixing of the two fluids and association of the biotinylated reagent 80 and any biotinylated cleavage products with the binding sites on the PPE-beads 82. Unreacted QTB will quench the fluorescence of the PPE-B while the cleaved biotinylated fragments will not.

A control is shown in FIG. 6B wherein no QTB is present in the upper compartment (i.e., 100% control). A control, for maximum quench is shown in FIG. 6C wherein no enzyme (e.g., protease) is added to the top compartment.

### EXAMPLE 3

The rapid hydrolysis of the QTB by enterokinase in solution can be taken advantage of in a homogeneous solution assay format wherein the PPE-B polymer and the QTB are complexed to Avidin molecules. By first complexing QTB to a small portion of the biotin binding sites present in a given amount of Avidin in solution followed by complexing binding sites remaining with PPE-B polymer, a soluble cross-linked QTP can be built. The fluorescence quench response can be tailored by varying the ratio of bound PPE-B polymer to bound QTB. Exposure of this polymer format to a sample containing enterokinase will result in an appropriate fluorescence recovery response, thus quantitating the enzyme.

### EXAMPLE 4

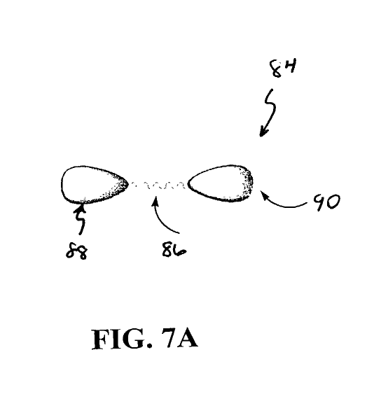

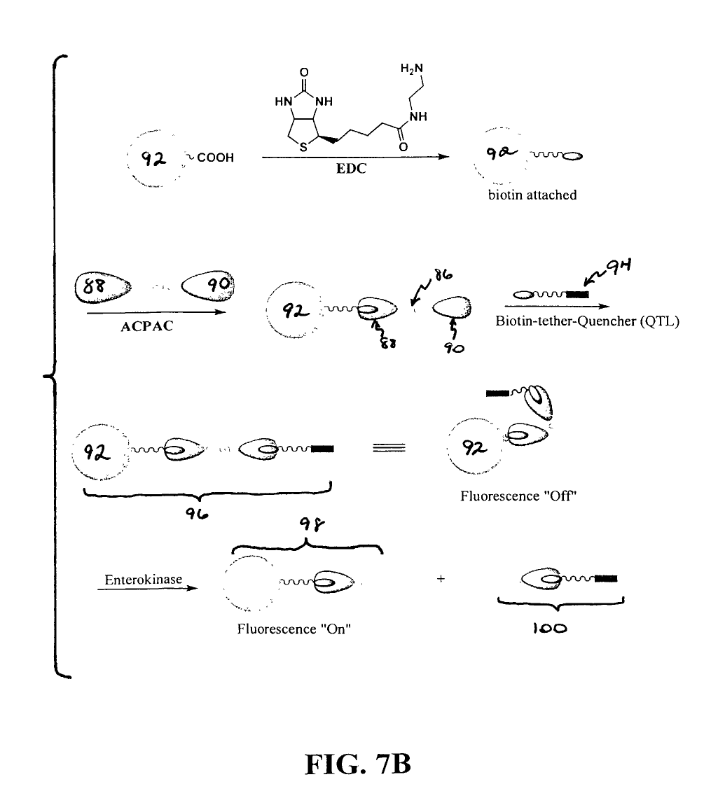

As shown in FIG. 7A, a plasmid construct 84 can be synthesized comprising a peptide tether 86 comprising an enterokinase cleavage site [e.g., (Asp)₄Lys] flanked by two avidin core binding cassettes 88 and 90. The peptide tether can, for example, comprise the following sequence

-Ala-Gly-Ser-Gly-Ser-Glu-Asp-Asp-Asp-Asp-Lys-Leu-Ala-Leu-.

The synthesis of a QTP bioconjugate from the plasmid construct 84 and the use of the QTP bioconjugate in an assay is illustrated in FIG. 7B. As shown in FIG. 7B, one of the binding cassettes 90 can be complexed to a biotinylated quencher (e.g., QSY-7) and the other binding cassette 88 can be conjugated to a biotinylated fluorescer (e.g., PPE polymer) to form a QTB bioconjugate according to the invention. First, a solid support 92 (a bead is shown) having surface carboxylic acid surface groups can be biotinylated using EDC. One of the avidin core binding cassettes 88 can then be reacted with the biotin group on the solid support. The remaining avidin core binding cassette can then be reacted with a QTB reagent 94 to form a QTP bioconjugate 96.

As shown in FIG. 7B, before cleavage of the tether, the fluorescence of the QTP bioconjugate 94 is quenched due to the proximity of the quencher and fluorescer. However, when the peptide tether is cleaved, the fluorescer containing fragment 98 and the quencher containing fragment 100 separate and fluorescence increases.

The reagent provides another soluble format for the QTP. This format enjoys the same advantage as the earlier formats in that varying the biotin loading density on the PPE polymer can control the sensitivity of response to enzyme in solution.

According to the invention, the amount of the target biomolecule (i.e., the target analyte) in the sample can be determined using known techniques. For example, a plurality of control samples containing different known amounts of target analyte can be incubated with the bioconjugate. The fluorescence of each of the incubated control samples can then be measured. A calibration curve can then be generated of fluorescence as a function of analyte concentration. The amount of analyte in a sample can then be calculated from the calibration curve.

From the foregoing, it will be appreciated that, although specific embodiments of the invention have been described herein for purposes of illustration, various modifications may be made without deviating from the spirit and scope of the invention.

What is claimed is:

1. A bioconjugate comprising:
a tether comprising a segment capable of recognizing and interacting with a target biomolecule;
a fluorescer comprising a plurality of associated fluorescent species, the fluorescer conjugated to a first location on the tether; and
a quencher for the fluorescer conjugated to a second location on the tether;
wherein the segment capable of recognizing and interacting with the target biomolecule is located between the first and second locations on the tether.

2. The bioconjugate of claim 1, wherein the segment capable of recognizing and interacting with the target biomolecule comprises a sequence selected from the group consisting of: a peptide; a nucleic acid; an oligosaccharide; and a glycoconjugate.

3. The bioconjugate of claim 1, wherein the fluorescer comprises a polymer or oligomer comprising a plurality of fluorescent repeating units.

4. The bioconjugate of claim 1, wherein the fluorescer comprises a solid support associated with a plurality of fluorescent species.

5. The bioconjugate of claim 4, wherein one or more quenchers are each linked to the solid support through a reactive tether.

6. The bioconjugate of claim 4, wherein the solid support is selected from the group consisting of: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes.

7. The bioconjugate of claim 1, wherein the the fluorescer is selected from the group consisting of: conjugated polyelectrolytes; biotinylated conjugated polyelectrolytes; functionalized conjugated oligomers; charged conjugated polymers; uncharged conjugated polymers; conjugated polymer blends; and J-aggregated polymer assembly comprising assembled monomers or oligomers.

8. The bioconjugate of claim 1, wherein the fluorescer is a virtual polymer.

9. The bioconjugate of claim 1, wherein the fluorescer is a poly(L-lysine) polymer or oligomer having cyanine pendant groups.

10. The bioconjugate of claim 1, wherein the fluorescer is constructed from an oligosaccharide.

11. The bioconjugate of claim 4, wherein the fluorescer comprises a fluorescent polymer or oligomer.

12. The bioconjugate of claim 11, wherein the fluorescent polymer or oligomer is associated with the solid support by: covalent attachment to the solid support; adsorption onto the surface of the solid support; or by interactions between a biotin moiety on the fluorescent polymer or oligomer and an avidin, neutravidin or streptavidin moiety on the solid support surface.

13. The bioconjugate of claim 1, wherein the fluorescer is conjugated to the tether via a protein molecule.

14. The bioconjugate of claim 13, wherein the protein molecule is selected from the group consisting of: avidin; neutravidin; and streptavidin.

15. A method of assaying for the presence and/or amount of a target analyte in a sample comprising:
incubating the sample with a bioconjugate as set forth in claim 1 wherein the tether comprises a segment capable of recognizing and interacting with the target analyte; and
measuring the fluorescence of the incubated sample;
wherein the measured fluorescence of the incubated sample is an indication of the presence and/or the amount of the target analyte in the sample.

16. The method of claim 15, further comprising:
measuring the fluorescence of a control containing no target analyte; and
comparing the fluorescence of the control to the fluorescence of the incubated sample;
wherein a difference in the fluorescence between the control and the incubated sample is an indication of the presence and/or the amount of the target analyte in the sample.

17. The method of claim 15, wherein measuring the fluorescence of the incubated sample comprises;
measuring the fluorescence of the incubated sample at a first time after incubation;
measuring the fluorescence of the incubated sample at a second time after incubation, the second time later than the first time; and
comparing the fluorescence at the first time to the fluorescence at the second time;
wherein a change in fluorescence from the first time to the second time is an indication of the presence and/or the amount of the target analyte in the sample.

18. The method of claim 15, further comprising:
incubating a plurality of control samples with the bioconjugate, each of the control samples comprising different known amounts of target analyte;
measuring the fluorescence of each of the incubated control samples;
generating a calibration curve of fluorescence as a function of analyte concentration from the measured fluorescence values of each control sample; and
calculating the amount of analyte in the sample from the calibration curve.

19. The method of claim 15, wherein the tether comprises an RNA segment and the target analyte comprises a nucleic acid, the method further comprising contacting the sample with an enzyme, wherein association comprises hybridization of the RNA segment of the tether and the target, the enzyme cleaving the hybridized RNA segment of the tether to produce a quencher containing bioconjugate fragment and a fluorescer containing bioconjugate fragment.

20. The method of claim 19, wherein the enzyme is ribonuclease H.

21. The method of claim 19, wherein the target hybridizes a plurality of bioconjugates.

22. The method of claim 15, wherein the segment capable of recognizing and interacting with the target analyte comprises a peptide sequence and the target analyte comprises an enzyme which can cleave the peptide sequence, cleavage of the tether resulting in formation of a quencher containing bioconjugate fragment and a fluorescer containing bioconjugate fragment.

23. The method of claim 22, wherein the enzyme is a protease, glycosidase, or transferase enzyme.

24. The method of claim 23, wherein the enzyme is a transferase enzyme which receives either the quencher containing bioconjugate fragment or the fluorescer containing bioconjugate fragment.

25. The method of claim 24, wherein the transferase enzyme is inactivated by the cleavage reaction.

26. The method of claim 25, wherein the cleavage reaction activates a secondary chemical process or initiates a catalytic chemical cascade.

27. The method of claim 15, wherein the segment capable of recognizing and interacting with the target analyte comprises an oligosaccharide or glycoconjugate sequence and the target analyte comprises an enzyme which can cleave the segment, cleavage of the segment resulting in formation of a quencher containing bioconjugate fragment and a fluorescer containing bioconjugate fragment.

28. The method of claim 27, wherein the enzyme is a glycosidase or transferase enzyme.

29. The method of claim 15, wherein the fluorescer comprises a solid support associated with a plurality of fluorescent species.

30. The method of claim 29, wherein one or more quenchers are each linked to the solid support through a reactive tether.

31. The method of claim 29, wherein the solid support is selected from the group consisting of: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes.

32. A method of assaying for the presence or amount of a target analyte in a sample comprising:
incubating the sample with a bioconjugate comprising a quencher and a reactive group conjugated to a tether at first and second locations respectively, wherein the tether comprises a segment between the first and second locations capable of recognizing and interacting with the target analyte;
adding a fluorescer to the incubated sample to form a sample mixture, the fluorescer comprising a solid support associated with a plurality of fluorescent species, wherein the solid support comprises surface functional groups reactive with the reactive group on the bioconjugate such that the bioconjugate can be attached to the solid support, the attachment of the bioconjugate to the solid support quenching the fluorescence of the fluorescer;
allowing the reactive group on the bioconjugate to react with the surface functional groups on the solid support; and
subsequently measuring the fluorescence of the sample mixture;
wherein the amount of fluorescence of the sample mixture indicates the presence and/or amount of the target analyte in the sample.

33. The method of claim 32, further comprising:
adding the fluorescer to a second sample that has not been incubated with the bioconjugate to form a control;
measuring the fluorescence of the control; and
comparing the fluorescence of the control to the fluorescence of the sample mixture;
wherein a difference in the fluorescence between the control and the sample mixture is an indication of the presence and/or the amount of the target analyte in the sample.

34. The method of claim 32, further comprising:
incubating a second sample containing no target analyte with the bioconjugate;
adding the fluorescer to the incubated sample to form a control;
measuring the fluorescence of the control; and
comparing the fluorescence of the control to the fluorescence of the sample mixture;
wherein a difference in the fluorescence between the control and the sample mixture is an indication of the presence and/or the amount of the target analyte in the sample.

35. The method of claim 32, further comprising:
incubating a plurality of control samples with the bioconjugate, each of the control samples comprising different known amounts of target analyte;
adding the fluorescer to each of the incubated control samples to form a plurality of control mixtures;
measuring the fluorescence of each of the control mixtures;
generating a calibration curve of fluorescence as a function of analyte concentration from the measured fluorescence values of each control mixture; and
calculating the amount of analyte in the sample from the calibration curve.

36. The method of claim 32, wherein measuring the fluorescence of the incubated sample comprises;
measure the fluorescence of the sample mixture at a first time after addition of the fluorescer;
measuring the fluorescence of the sample mixture at a second time after addition of the fluorescer, the second time later than the first time; and
comparing the fluorescence of the sample mixture at the first time to the fluorescence of the sample mixture at the second time;
wherein the fluorescence of the sample mixture at the first time compared to the fluorescence of the sample mixture at the second time is an indication of the presence and/or the amount of the target analyte in the sample.

37. The method of claim 32, wherein the change in fluorescence results from cleavage of the tether.

38. The method of claim 32, wherein the target analyte is an enzyme.

39. The method of claim 32, wherein the bioconjugate is biotinylated and the surface of the solid support comprises avidin, neutravidin or streptavidin.

40. An intracellular assay comprising:
transfecting a cell with a bioconjugate as set forth in claim 1 wherein the target analyte is an intracellular target analyte; and
measuring the fluorescence of the cell;
wherein the measured fluorescence of the cell indicates the presence and/or amount of the target analyte in the cell.

41. The assay of claim 40, wherein the fluorescer is a poly(L-lysine) polymer having cyanine pendant groups.

42. The assay of claim 40, wherein the fluorescer is constructed from an oligosaccharide.

43. The assay of claim 40, wherein the target analyte is an enzyme.

44. The assay of claim 40, wherein the target analyte is a caspase enzyme the presence of which in the cell indicates the initiation of apoptosis.

45. A biotinylated fluorescer comprising a plurality of associated fluorescent species.

46. The biotinylated fluorescer of claim 45, wherein the biotinylated fluorescer comprises a biotinylated fluorescent polymer or oligomer.

47. The biotinylated fluorescer of claim 45, wherein the biotinylated fluorescer comprises a biotinylated poly(phenylene ethynylene) polymer.

48. The biotinylated fluorescer of claim 45, wherein the biotinylated fluorescer comprises a biotinylated solid support having a plurality of fluorescent species associated therewith, wherein the solid support comprises biotin groups available for reaction.

49. The biotinylated fluorescer of claim 48, wherein the solid support is selected from the group consisting of: streptavidin coated spheres; polymer microspheres; silica microspheres; organic nanoparticles; inorganic nanoparticles; magnetic beads; magnetic particles; semiconductor nanoparticles; quantum dots; membranes; slides; plates; and test tubes.

50. A quenching reagent comprising:
a tether comprising a segment capable of recognizing and interacting with a target biomolecule;
a quencher conjugated to a first location on the tether, the quencher capable of quenching the fluorescence of a fluorescer comprising a plurality of associated fluorescent species; and
a biotin molecule conjugated to a second location on the quencher;
wherein the segment capable of recognizing and interacting with the target biomolecule is located between the first and second locations on the tether.

51. The quenching reagent of claim 50, wherein the segment capable of recognizing and interacting with a target biomolecule comprises a peptide sequence.

52. A bioconjugate comprising:
a tether comprising a segment capable of recognizing and interacting with a target biomolecule;
a first avidin molecule conjugated to a first location on the tether; and
a second avidin molecule conjugated to a second location on the tether;
wherein the segment capable of recognizing and interacting with the target biomolecule is located between the first and second locations on the tether.

53. The bioconjugate of claim 52, wherein the segment capable of recognizing and interacting with a target biomolecule comprises a peptide sequence.

54. The bioconjugate of claim 52, further comprising:
a fluorescer comprising a plurality of associated fluorescent species conjugated to the first avidin molecule; and
a quencher capable of quenching the fluorescence of the fluorescer conjugated to the second avidin molecule.

55. The bioconjugate of claim 54, wherein the fluorescer is a solid support having a plurality of fluorescent species associated therewith.
